(19) United States
(12) Patent Application Publication
(10) Pub. No.: US 2026/0196333 A1
(43) Pub. Date: Jul. 9, 2026

(54) METHOD OF MAPPING COGNITIVE TASKS TO BRAIN REGIONS TO IMPROVE COGNITIVE THERAPY

(71) Applicant: Clarity Technologies SASU, Paris (FR)

(72) Inventors: Carolina Reis, Paris (FR); Raphael Certain, Paris (FR)

(21) Appl. No.: 19/445,383

(22) Filed: Jan. 9, 2026

Related U.S. Application Data
(60) Provisional application No. 63/743,571, filed on Jan. 9, 2025.

(51) Int. Cl. G16H 20/70; A61M 21/00; A61M 21/02; G06F 3/01; G16H 50/20

(52) U.S. Cl. CPC G16H 20/70; A61M 21/02; G06F 3/015; G16H 50/20; A61M 2021/0027; A61M 2021/005; A61M 2209/088; A61M 2230/10

(57) ABSTRACT

A method and system are disclosed for delivering neuromodulation therapy within a virtual-reality environment by dynamically mapping cognitive task engagement to activation of targeted brain regions. The system presents immersive virtual-reality environments and cognitive tasks to a user while monitoring physiological signals indicative of neural activity during task performance. Based on an analysis of the monitored neural activity, the system determines whether a targeted brain region is activated and selectively adapts therapeutic delivery by modifying task selection, virtual-reality content, or neuromodulation protocols. Neuromodulation may include visual, auditory, audio-visual, or electrical stimulation integrated within the virtual-reality environment and synchronized with task execution. By explicitly linking task-evoked activation of specific brain regions to adaptive selection and delivery of neuromodulation within an immersive virtual-reality environment, the disclosed approach addresses the challenge of achieving precise, responsive, and engaging neural modulation in therapy.

### CROSS-REFERENCE TO RELATED APPLICATION

This application claims the priority benefit of U.S. provisional patent application No. 63/743,571 filed Jan. 9, 2025, which are incorporated by reference herein in their entirety.

### FIELD OF THE DISCLOSURE

The present disclosure is generally related to mapping cognitive tasks to brain regions to improve cognitive therapy.

### BACKGROUND

With 90 million deaths and 16% of global annual deaths, neurological disorders are the second leading cause of death after heart disease and the leading cause of disability. Neurological disorders include stroke, spine injuries, epilepsy, sleep disorders, Alzheimer's disease, Parkinson's disease, dementia with Lewy bodies, primary progressive aphasias, frontotemporal dementia, corticobasal syndrome, progressive supranuclear palsy, posterior cortical atrophy, demyelination, and others. In these diseases, the cognitive ability of the patients is impaired and may also decline over time. As the population ages, the number of people with neurological disorders is expected to increase and become a heavy burden on society and healthcare systems. With more than 46.8 million people worldwide, dementia is one of the most devastating neurological disorders. The number of patients is expected to increase to 74.7 million by 2030 and 131.5 million by 2050. Alzheimer's disease causes more than 60% of dementia cases. Among the neuropathophysiological features associated with Alzheimer's, one can generally observe an increase in amyloid-beta protein, hyperphosphorylation of tau protein, inflammatory activity of microglia, and dendritic tree degeneration. It has also been shown that neural oscillations, particularly theta-gamma coupling, are disrupted in Alzheimer's and that these impairments manifest before any behavioral deficit.

Alzheimer's disease is a progressive neurodegenerative disorder characterized primarily by abnormal amyloid-beta deposition and tau hyperphosphorylation. Additionally, neuroinflammation plays a pivotal role in Alzheimer's, involving the activation of immune cells such as microglia and astrocytes, leading to the release of pro-inflammatory cytokines, chemokines, and other inflammatory mediators. Clinically, Alzheimer's is the predominant cause of dementia, a broad term encompassing severe and persistent cognitive decline, notably affecting memory. Despite the widespread prevalence and profound impact of this neurological disorder, there is currently no therapeutic intervention for Alzheimer's that guarantees both a heightened safety profile and substantial clinical advantages. This situation extends to the recently approved anti-amyloid immunotherapies, which, despite possessing disease-modifying attributes for Alzheimer's disease, exhibit marginal clinical efficacy, along with considerable costs and notable adverse effects such as cerebral swelling and hemorrhage. Consequently, there is an imperative need for therapeutic alternatives for those suffering from Alzheimer's disease.

Abnormalities in neuronal activity may directly contribute to the pathogenesis of the disease. Neural oscillation includes the rhythmic or repetitive neural activity in the central nervous system, such as oscillations in membrane potential or rhythmic patterns of action potentials. Synchronized activity of neurons can result in macroscopic oscillations, which can be observed via electroencephalography (EEG). Sensory stimulation is the precise delivery of sensory stimuli, such as visual, auditory, or mechanical stimulation, at specific frequencies to influence the firing patterns of sensory brain regions and downstream areas like the memory-centric hippocampus. This non-invasive neuromodulation technique may potentially treat Alzheimer's by modulating cognition-related electrical activity patterns of specific brain regions and consequently delaying neuro and cognitive degeneration. Research suggests that chronic exposure to sensory stimulation at gamma frequencies, more specifically to 40 Hz, can safely delay the progression of Alzheimer's. The persistent application of 40 Hz audiovisual stimulation to users experiencing mild to moderate dementia due to Alzheimer's may engage neuroprotective mechanisms with the potential to slow the progression of the disease. Examples of slowed progression may include reduced whole-brain, cortical, and hippocampal atrophy, decreased ventricular enlargement, and strengthening of connectivity in the default mode network, and in the medial temporal lobe-prefrontal cortex network.

Gamma sensory stimulation targets neurodegenerative mechanisms common in Alzheimer's and normal aging, such as beta-amyloid accumulation, neuroinflammation, and neural loss. Gamma frequencies are those in the range of 30-100Hz. Sensory stimulation seeks to achieve neural entrainment, referring to the process by which brain activity synchronizes to the frequency of external stimuli. Gamma sensory stimulation may be delivered via audio and/or visual stimuli. Gamma sensory stimulation may be delivered via an augmented reality (AR), mixed reality (MR), or virtual reality (VR) headset. It may also include contextually-rich sensory stimulation, wherein sensory inputs are integrated into environments requiring one or more cognitive processes, such as memory encoding, memory consolidation, memory recall, perception, attention, knowledge formation, problem-solving, emotion processing, concept formation, pattern recognition, association, decision making, motor coordination, task planning, language expression, language comprehension, etc. Cognitive tasks may improve entrainment, particularly by involving regions of the brain that may otherwise be unaffected by audio and/or visual stimuli alone. Cognitive tasks may be mapped to treatable symptoms to facilitate the creation of treatment plans using historical data to identify correlations between symptoms and cognitive tasks, which improved patient outcomes when paired with sensory stimulation.

Improved patient outcomes may include improvements in behaviors, including anxiety, depression, addiction, and sleep. Additionally, patients may see improvement in one or more cognitive skills, including perceptual reasoning, sustained attention, selective attention, divided attention, long-term memory, working memory, logic and reasoning, auditory processing, visual processing, visual-motor planning and processing, visual-spatial planning and processing, auditory memory, visual memory, task planning, task sequencing, task initiation, task completion, visual encoding and decoding, auditory encoding and decoding, sensory encoding and decoding, language expression, language comprehension, processing speed, emotion processing, cognitive control, cognitive inhibition, declarative memory, procedural memory, episodic memory, auditory memory, visual memory, semantic memory, or autobiographical memory. These cognitive skills may additionally comprise cognitive tasks which may be paired with a stimulation treatment. Additional benefits may include improvements in visual, language, auditory, or motor processing speeds.

Cognitive tasks may comprise a mental component, such as learning, presenting, speaking, analyzing, listening, etc. Cognitive tasks may additionally comprise a physical component, including physical activities such as walking, jogging, skipping, running, swimming, etc. Cognitive tasks may include playing a logic game, board game, or video game; or interacting with an environment with gamified elements such as rewards for completing specific actions or engaging with emotionally charged content. Stimulation treatments may additionally be paired with pharmacological interventions or may be used to treat the side effects of medications. The cognitive tasks discussed above integrate with the claimed invention as an additional embodiment, such as mapping the neural response to cognitive tasks performed within virtual reality environments to identify and adjust stimulation parameters, such as frequency and intensity, dynamically targeting specific brain regions to enhance therapy for neurodegenerative diseases. The stimulation may be provided as a pure sine wave or a rectangular wave. In some embodiments, the frequency used may be any frequency between 0.1 Hz and 200 Hz. Variable parameters may include frequencies, waveforms, stimuli intensity, etc. The effectiveness of stimuli may also be evaluated using cognitive tests administered before and after stimulation treatment. Cognitive tests may be delivered after the treatment and in relation to a baseline (e.g., cognitive score at the very beginning, before receiving the medical device). A correlation may exist with Alzheimer's sleep disorders, particularly excessive nocturnal awakenings, which may be improved via gamma sensory stimulation. Actigraphy recordings during nighttime may be used to monitor sleep quality, identify sleep disorders, and determine the effectiveness of treatments. They may also correlate specific neural activity patterns captured via EEG.

Sensory stimulation may be described as oscillations, pulses, pulse trains, flashes, etc. The sensory stimulation may target specific frequency ranges, and different modalities may be used separately or in combination, including visual, auditory, mechanical, electrical, tactile, magnetic, etc. Different sensory stimulation modalities may be coordinated such that the frequencies are synchronized or specific frequencies are ordered via specific modalities in an intentional manner. For example, a light may pulse at 40 Hz while an auditory signal at 4 Hz is administered. Alternatively, visual and auditory stimulation may be alternated rather than occurring simultaneously. Electrical or magnetic stimulation, such as transcranial electric stimulation or transcranial magnetic stimulation, may alternatively be utilized. Stimulation may also be delivered continuously without interruptions or with purposeful interruptions as part of the therapeutic process in order to for instance engage the patient into prediction computations to which the hippocampus plays a key role.

Visual stimulation may be administered at a target frequency with a minimum contrast level. The image or background brightness may be measured in lumens, footcandles, lux, or nits, depending on the light source. An example of a minimum level of contrast may be a minimum of 100 nits, such that the active region of a display may be required to be at least 100 nits higher than the background or inactive areas. Similarly, audio stimulation may be necessary to exceed a threshold or amount above a noise floor. Visual and auditory stimulation frequencies may be additive or may alternatively be subtractive. For example, ambient light may be used with an electrochromic lens capable of oscillating state at the target frequency. Alternatively, an audio frequency may be modulated to achieve a desired frequency pattern, which may include adding or removing frequencies. The visual stimulation discussed above integrates with the claimed invention as an additional embodiment, such as providing a visual stimulation that is linked, associated, or corresponds to a certain brain region. In addition to the treatment of Alzheimer's and sleep disorders, sensory stimulation may provide treatment options for other neurological diseases such as multiple sclerosis, Parkinson's disease, epilepsy, spinal injuries, stroke, primary progressive aphasias (PPA), frontotemporal dementia, corticobasal syndrome, progressive supranuclear palsy, and posterior cortical atrophy. Sensory stimulation may also be used to treat anxiety, depression, and dementia occurring from natural aging processes. Sensory stimulation may achieve benefits including blood vessel dilation, glial responses, reduction in beta-amyloid deposition via activation of the glymphatic system, improved cerebrospinal fluid flow, and reduced brain atrophy.

The sensory stimulation may be provided by a virtual reality headset. The virtual reality headset comprises at least one screen capable of displaying one or more images to a user's eyes. In some embodiments, the images may be delivered sequentially at a fixed speed or framerate. For example, the frame rate may be 80 frames per second or 80 Hz. In some embodiments, the frame rate may be variable. Examples of displays may utilize technologies including liquid crystal displays (LCD), organic light-emitting diodes (OLED), micro LED, etc. The virtual reality headset discussed above integrates with the claimed invention as an additional embodiment, such as providing the sensory stimulation to a certain brain region of the patient to enhance the therapy session.

A virtual reality headset may refer to an augmented reality headset. An augmented reality headset may include one or more cameras that can pass through video of the environment in front of the user or may utilize a semitransparent or transparent display that allows the user to see beyond the digital image. In some embodiments, the displays may comprise diffractive waveguides, reflective waveguides, transparent OLED displays, etc. Augmented reality headsets may use camera data to modify a digital image based on the environment. For example, a label may be superimposed on an object within the user's field of view. The augmented reality headset discussed above integrates with the claimed invention as an additional embodiment, such as providing the sensory stimulation to a certain brain region of the patient to enhance the therapy session.

Sensors may be directly integrated into the virtual reality headset or may be discrete and optionally used in parallel with the virtual reality headset or separately to assess responsiveness to the stimuli. Sensors may measure physiological parameters, including heart rate, pupillometry, eye movement, electrodermal conductance, body temperature, respiratory rate, and heart rate variability. Examples of sensors may include a pulse oximeter, electroencephalogram (EEG), electromyography (EMG) sensor, galvanic skin response sensor, magnetic resonance imaging sensor (MRI), electrocorticography (ECoG), etc. EEG may be used to monitor a user's brain activity using a plurality of electrodes. In some embodiments, brain activity may be measured using functional near-infrared spectroscopy (fNIRS), which estimates neural activity based on cortical hemodynamic activity. Measurement of brain activity may include measurement of brainwave phase and/or amplitude. Sensors may be used to identify symptoms and conditions that may be treated with sensory stimulation, which may consist of mapping symptoms to treatments comprising at least a target stimulation frequency and modality.

Sensors may comprise one or more cameras, which may be aimed away from the user to monitor the environment and may be used to provide pass-through imagery of the user's surroundings. Cameras may alternatively be aimed toward the user, such as at the user's eyes, to track the user's eye and facial movements. In some embodiments, cameras and other sensors may be used to track the user's location and movements within their physical environment.

Sensors may be used to determine the user's level of attention and which virtual or physical objects the user is looking at or focused on. The sensors may additionally measure engagement with objects, the environment, or a task. These measurements may be used in assessments or to enable gamification, such as rewarding a user based on their level of engagement with a task. Sensor measurements may additionally be used to adapt the virtual environment to the user. The sensors discussed above integrate with the claimed invention as an additional embodiment, such as monitoring a specific brain region to determine if the cognitive task provided to the patient has activated the desired brain region.

A virtual reality headset may further comprise a communication interface to communicate with a computing device. The communication interface may be wired, such as Ethernet, USB, HDMI, DisplayPort, or other hardwired connections. Alternatively, the communication interface may be wireless, such as Wi-Fi, Bluetooth, LTE, 3G, 4G, or 5G cellular. The interface may utilize a transmission protocol that utilizes compression algorithms to facilitate efficient communication between a computing source device and the virtual reality headset. The communication interface discussed above integrates with the claimed invention as an additional embodiment, such as allowing the response module 110, recording module 112, and mapping module 114 to transmit data to and from the headset 128.

A virtual reality headset may include one or more speakers or other sound-emitting devices positioned near the ear when worn by a user. Sound-emitting devices, might also include bone conduction headphones, which transmit auditory input through vibratory stimulation of the cranial bones to directly stimulate the cochlea. The speakers may emit sounds toward a user's ears and may include features such as spatial audio, noise canceling, etc., to direct sound toward the user. In some embodiments, the speakers may be in external headphones or earbuds. The virtual reality headset may include one or more microphones, which may be used to monitor the environment around the user, receive feedback regarding the treatment, and facilitate communication with other users, caregivers, or a remote physician. The speakers discussed above integrate with the claimed invention as an additional embodiment, such as providing an audio stimulus that corresponds or is associated with a specific brain region to enhance the therapy session.

Collected sensor data may be analyzed and transmitted to a physician or caregiver to provide a user's status. Data may further indicate the user's compliance with a treatment plan. A physician or caregiver may be able to customize or alter a treatment plan remotely from a secondary device. The treatment plan may further be modified dynamically. The sensor data discussed above integrates with the claimed invention as an additional embodiment, such as determining if the desired brain region of the patient is progressing or regressing and/or therapeutic efficacy.

The collected sensor data may further be aggregated within a database. The data may be used to train machine learning and artificial intelligence models. Trained models may be used to provide a user status based on collected data, dynamically adjust a treatment, assess whether a treatment is indicated, or determine whether a treatment should be stopped. Sensor data may be correlated to mood, which may facilitate the identification of a user's mood and positive and negative mood triggers. Sensor data may additionally be used to monitor a user's sleep quality. Trained models may also be used to provide feedback to users and caregivers, which may be used to impact treatment or other behaviors. For example, a model may indicate that a user's sleep quality is higher when they eat two hours before going to sleep or that a user performs better on memory tasks when they sleep at least 9 hours each night.

A machine learning or artificial intelligence model may be used to identify whether a user may benefit from sensory stimulation therapy and may further recommend one or more treatment parameters. A similar model may be used to determine the effectiveness of the sensory stimulation treatment and whether the treatment should continue or be stopped. The models may be located on a headset or may access a secondary device or server. These assessments may be conducted periodically, on an ongoing basis as data is collected, or may be manually initiated at the discretion of a physician, patient, or caregiver.

The assessments may utilize passively collected data or may additionally comprise guided tests comprising of prompts the user must follow and providing a score relating to the user's performance. The score may be compared to a previously obtained score to determine the user's progress and the effectiveness of the treatments. A machine learning or artificial intelligence model may also be used to predict a user's response to a sensory stimulation treatment, which may be used to determine whether such therapies should be initiated or continued. Externally collected data, such as from an EEG or MRI, may further be used for such assessments. Assessments and algorithms other than machine learning or artificial intelligence models may also be used to evaluate progress and create treatment plans. The machine learning or artificial intelligence models discussed above integrate with the claimed invention as an additional embodiment, such as analyzing collected patient data to determine if a specific brain region is activated through the cognitive task that was provided.

The virtual reality headset may present a virtual reality environment to a user. The virtual reality headset may access a library of virtual environments and/or stimulation protocols. The virtual reality environments and/or stimulation protocols may be adaptive, such that they are dynamically responsive to sensor data, the user's actions, etc. The virtual reality environments and/or stimulation protocols may be selected based on the user's measured physiological parameters.

The virtual environment may be customized based on data specific to the user, including images or videos, including people, places, or things relevant to or known to the user. The virtual environment may be configured to facilitate at least visual or auditory sensory stimulation. The virtual environment may mimic places familiar to the user or may alternatively comprise features new to the user. The virtual environment may include elements that are impossible or impractical to replicate in the physical world. The virtual environment may additionally replicate weather, seasonality, time of day, etc.

The virtual environment and/or treatment plan may include customizations for the user, including considerations such as age, gender, dominant hand, lived experiences, etc. The virtual environment may simulate specific times, which may correspond to a time in the real world or may intend to evoke an additional response, such as tiredness if used before bedtime to improve the user's sleep quality. The virtual environment may also be generated with consideration of the user's symptoms, including those being treated and those that may be untreatable. For example, nicotine and caffeine use, as well as poor sleep, memory, and cognitive function, may be treatable via sensory stimulation. Vision and hearing impairment may not be treatable; however, it may impact the effectiveness of the treatment. The virtual environment discussed above integrates with the claimed invention as an additional embodiment, such as providing stimulation protocols that are associated with specific brain regions to target areas of the brain of the patient that require treatment.

The virtual reality environments may be interactive, receiving user feedback, such as via eye gaze tracking. In some embodiments, eye tracking may enable a gamified experience to improve user engagement. The user may be presented with a cognitive task and is rewarded when the task is complete. These tasks may further guide a user's attention to specific regions within their field of view, which may correspond with a location where stimulation is provided. Cognitive tasks may include tracking or interacting with objects. Tasks may include guided meditation and neurofeedback protocols, and the user may be monitored for compliance with the provided instructions. The impact may be measured by sensor parameters, including heart rate, respiratory rate, etc. The cognitive tasks discussed above integrate with the claimed invention as an additional embodiment, such as providing a plurality of cognitive tasks to the patient to activate a specific brain region and then delivering a stimulation protocol that is associated with that cognitive task to provide a stimulation protocol that is effective for a specific region of the brain.

Sensory stimulation may be provided as an auditory or visual pattern within a range of 1 Hz to 120 Hz and may include frequencies corresponding to neural oscillations in the delta (1-4 Hz), theta (4-7 Hz), alpha (8-13 Hz), beta (14-30 Hz), and gamma (30-100 Hz) ranges. In an embodiment, the stimuli may be provided at a frequency of 40 Hz, which has been associated with higher cognitive functions such as memory, attention, and perception. To provide a stimulus utilizing light, such as via the display of a virtual reality headset, the display's refresh rate may be required to be set at a multiple of the target stimuli frequency, such as double the intended frequency. For example, a stimulus with a frequency of 40 Hz would require a frame rate of 80 Hz or 120 Hz. In some embodiments, the refresh rate may be selected as the highest whole number multiple of the target frequency within the capability of the hardware. For example, if the maximum refresh rate of a display is 150 Hz, the refresh rate of the display may be set to 120 Hz for a stimuli frequency of 40 Hz, as 3 is the largest whole number multiple of 40 in 150. The refresh rate and the rate at which an application generates frames to be displayed may be required to be constant. In some embodiments, the target stimuli and/or refresh rate may be variable. The sensory stimulation discussed above integrates with the claimed invention as an additional embodiment, such as delivering the sensory stimulation that is targeted or mapped to specific brain regions.

Theta stimulation can have similar effects as gamma stimulation on cognition as it targets different neural structures, such as the hippocampus, deeper within the brain. Stimulation at theta frequencies might induce beneficial effects on memory-related processes by inducing theta neural entrainment in different target neural structures depending on the cognitive task it is being paired with. Alzheimer's patients have been observed with reduced brainwave levels in both the theta and gamma frequency ranges. Theta stimulation has demonstrated the potential to increase memory recall using audio and/or visual oscillations at frequencies of 4-7 Hz during a cognitive task, particularly those involving predictions and memory. Theta frequencies may be used with gamma frequencies to achieve a synergistic effect. In an embodiment, this may be achieved by nesting waves at 4 Hz with waves at 40 Hz. The wave types of the different frequencies may be the same wave type or may be different, such as one being sinusoidal and the other being an isochrone wave. Nested waves may further have a zero or non-zero phase offset. The theta stimulation discussed above integrates with the claimed invention as an additional embodiment, such as providing theta stimulation to target the hippocampus for a patient who is struggling with memory cognitive tasks.

A visual stimulus may comprise an active area and an inactive area. An active area is an area of a display or a region of a user's field of view in which a stimulus is provided. An inactive area is an area of a display or region of the user's field of view in which a stimulus is not provided. For example, an active area may comprise a display region in which a white square is flashed at a target frequency. Alternatively, an active area may comprise bright colors or a region of high luminance or contrast. In some embodiments, an active area may comprise an LED or array of LEDs shining light within a user's field of view oscillating at a target frequency.

Larger, brighter, and higher contrast between active and inactive areas, or the oscillating states of an active region, may improve entrainment and overall effectiveness of the sensory stimulation; however, the same properties may be uncomfortable for a user receiving the treatment. Using different colors with less contrast than black and white may still be effective with less discomfort and possibly improved immersion. Similarly, border contrast may not need to be sharp. Images or video content may be layered on top of the active area or present elsewhere in the user's field of view. In some embodiments, the active areas may be embedded within regions of an image or video content or may modify parts of video content, such as changing the flashing pattern of light within the video to match a target frequency of 40Hz. In other embodiments, the entire field of view may be an active area, with the whole video or image oscillating between high and low luminance levels at a target frequency.

Effective sensory stimulation may require an active area to comprise a minimum percentage of a user's field of view, such as 25%. The minimum effective size of an active area may vary by user. Likewise, visual stimuli may be more effective when the active region comprises specific color components, such as red. In some embodiments, specific color combinations for active regions and inactive regions may result in effective stimulation while achieving an imperceptible flicker. Sensory stimulation effectiveness may be assessed based on the level of entrainment, which may be measured by EEG, cognitive assessments, or brain atrophy, as measured by MRI.

The location of a visual stimulus may additionally impact its effectiveness. The stimulus may be most effective within the fovea, or 1% of the central visual field; however, in some embodiments, the stimulus may be effective within 22 degrees of a user's field of vision. Such adaptation might happen in real-time or from a session to another. For instance in the case where sensory stimulation is embedded in a neurofeedback protocol where a threshold in the 40 hz power measured via occipitoparietal EEG distinguishes a good from a bad level of entrainment and adapts the environment (here the flickered vision filed) in real-time. Alternatively, if after a therapeutic session, the individual performed poorly in a cognitive assessment, in the next session the visual field might be adjusted. Likewise, the distance of the stimulus to the user may impact effectiveness, with closer distances being more effective and further distances being less effective.

Sensory stimulation may be provided via an audio signal by applying a 40 Hz isochronic tone to an original audio source, such as a carrier frequency. Stimulation may also be sinusoidal waveforms. Audio may be modulated via any of, amplitude modulation, frequency modulation, or isochrone modulation. An audio stimulus may be coordinated with a visual stimulus such that the same frequency, wavelength, and amplitude of the audio stimuli are matched to the corresponding visual stimuli. Video content may be generated, synchronized, and then queued before being displayed to ensure the video and audio stimuli are synchronized. In some embodiments, video content may be generated in parallel, including redundant frames such that one or more frames may be selectively dropped to ensure the desired synchronization and stimuli frequencies are achieved. Similarly, a component of content and/or a stimulus may be delayed to ensure the stimuli components are aligned. The audio stimulus discussed above integrates with the claimed invention as an additional embodiment, such as providing an audio stimulus that targets a specific region of the brain to enhance treatment. The way the sound is presented may vary in addition to the frequency. For example, in short trains that have a predictable pattern versus no pattern, i.e., presented randomly), where the former and not the latter are expected to target specific regions (like the hippocampus).

Sensory stimulation may additionally comprise peripheral nerve stimulation, such as may be achieved by placing electrodes on the surface of a user's skin. For example, electrodes may be placed on the user's neck or forearm and risk to target specific nerves. Electrical stimulation may then be applied via the electrodes to provide further stimulation, which may be synchronized with audio and/or visual stimulation. Peripheral nerve stimulation may be effective in improving psychomotor impairments resulting from neurodegenerative disorders such as Parkinson's disease, essential tremor, and Alzheimer's. In some embodiments, electrodes may be implanted within a user's body. The sensory stimulation discussed above integrates with the claimed invention as an additional embodiment, such as providing sensory stimulation that is designed to affect a specific brain region to enhance the patient's treatment.

Sensory stimulation may be delivered in combination with multimedia content of neutral or non-neutral valence. Non-neutral content, in particular, is expected to engage limbic and memory systems. Pairing stimulation with emotionally charged content is expected to amplify the magnitude and spread of neural entrainment to flicker rates not only in sensory regions but also in deep brain structures. Content might therefore be generated and rated based on its emotional valence by the patient, ensuring alignment with their personal preferences and responses. Additionally, machine learning algorithms may analyze the patient's ratings and responses to suggest novel content that is emotionally relevant and tailored to their specific needs, dynamically adapting to improve therapeutic efficacy. The personalized sensory stimulation discussed above integrates with the claimed invention as an additional embodiment, such as utilizing emotionally rated multimedia content to target specific brain regions, thereby enhancing therapeutic outcomes.

Parameters of sensory stimulation may be adjusted to personalize a treatment to a specific user. Examples include the stimulation frequency, size or location of the active area, contrast or color, brightness, types of stimulation, etc. For example, while 40 Hz may be the average target frequency, a user's EEG data may indicate improved entrainment at 38 Hz; therefore, the personalized target frequency for the user may be 38 Hz. Likewise, a user may be unable to tolerate the contrast of an active area oscillating between white and black, and therefore, different colors may be used in the active region, such as red and blue, while maintaining adequate contrast. The personalized sensory stimulation discussed above integrates with the claimed invention as an additional embodiment, such as providing personalized sensory stimulation that is also associated with a specific brain region to provide the most effective targeted treatment for a patient.

Sensory stimulation treatments may comprise daily treatment sessions over an extended period. For example, treatments may last 30-60 minutes and may be repeated daily for 3-12 months. The session durations may vary from a few seconds or a minute to multiple hours, such as the duration of a movie or other video and/or audio content. Likewise, the total treatment period may be as short as a single session, several days, weeks, months, or multiple years. Sessions may not be required daily but instead may be required on alternating days or less frequently. Session length and length of the treatment period may be customized to each user based on each user's symptoms, feedback, sensor measurements, and analysis of collected data. The sensory stimulation treatments discussed above integrate with the claimed invention as an additional embodiment, such as determining the sensory stimulation treatments based on the brain region being targeted.

Collected data may include device performance and usage, cognitive and neural biomarkers, attention levels, cognitive assessments, mood questionnaires, etc. The collected data may be used to customize a treatment plan and/or determine when to stop treatment. In some embodiments, the data may be used to calculate a cognitive score for a user. Cognitive scores may include any sleep score, memory score, attention score, motor ability score, etc. Physicians and caregivers may use the collected data to manage compliance with a treatment plan. Likewise, insurance companies may use the data to manage compliance and verify claims. The device and collected data may be accessed remotely, such as by a physician remotely uploading or modifying a treatment. The collected data discussed above integrates with the claimed invention as an additional embodiment, such as using the collected data to determine if a specific brain region was activated or not and, if so, sending a stimulation that corresponds to the cognitive task presented to the user to target that specific brain region.

Sensory stimulation therapies may be used to treat or mitigate symptoms of a wide range of diseases and conditions resulting from injuries. These conditions include frontotemporal dementia, chronic traumatic encephalopathy, corticobasal degeneration, genetic disorders such as inherited ataxia, chronic traumatic encephalopathy, stroke, and related cerebrovascular diseases. It may similarly alleviate symptoms of demyelinating diseases associated with brain atrophy, such as multiple sclerosis or acute disseminated encephalomyelitis. Sensory stimulation may slow the progression or help reduce the risk of such conditions developing. It may additionally benefit individuals experiencing stress and difficulty sleeping, in addition to cognitive performance such as perception, attention, and memory, which may help the performance of everyday tasks.

Improved cognitive capacity may be achieved by alleviating symptoms of brain atrophy such as a loss of neurons, memory loss, blurred vision, aphasia, impaired balance, paralysis, decreases in cortical volume, increases in cerebral spinal fluid volume, loss of motor control, difficulty speaking, comprehension, memory, decrease in gray and/or white matter, decrease in neuronal size, loss of neuronal cytoplasmic proteins, or combinations of such symptoms. These symptoms may alternatively be delayed or prevented.

### BRIEF DESCRIPTIONS OF THE DRAWINGS

FIG. 1: Illustrates a mapping of cognitive tasks to brain regions to improve cognitive therapy, according to an embodiment.

FIG. 2: Illustrates a Response Module, according to an embodiment.

FIG. 3: Illustrates a Recording Module, according to an embodiment.

FIG. 4: Illustrates a Mapping Module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

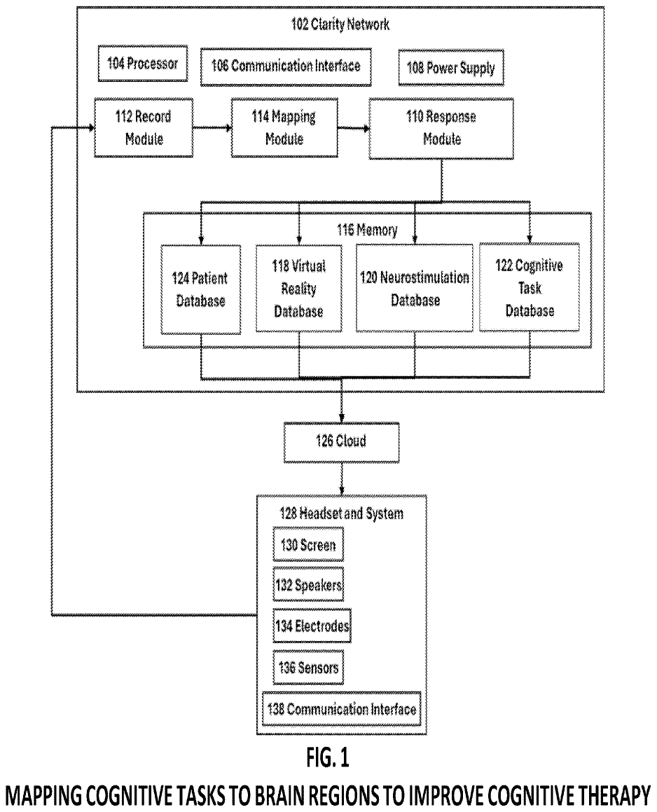

FIG. 1 illustrates a method for mapping cognitive tasks to brain regions to improve cognitive therapy. This method comprises a clarity network 102 that enables the integration of patient performance analysis, recording, and mapping processes with comprehensive databases for cognitive tasks, neurostimulation protocols, and virtual reality environments to deliver tailored neurostimulation therapy for neurodegenerative diseases. The clarity network 102 contains a response module 110, a recording module 112, and a mapping module 114. The response module 110 initiates VR tasks and stimulation protocols based on feedback from the mapping module 114, while the recording module 112 captures and stores performance and physiological data in the patient database 124 for further analysis. The mapping module 114 analyzes patient data to determine if the desired brain regions are activated and dynamically selects or adjusts VR tasks and stimulation protocols based on neural activity. The clarity network 102 includes a virtual reality database 118, which stores immersive VR environments designed to engage specific brain regions, a neurostimulation database 120, which provides stimulation protocols targeting therapeutic goals, and a cognitive task database 122, which contains a library of cognitive, motor, and functional tasks aligned with neural activation requirements. The patient database 124 maintains patient-specific data, including performance metrics, neural activity patterns, and session histories, to guide personalized therapy. The clarity network 102 connects with the headset 128, which delivers VR content, cognitive tasks, and synchronized stimulation protocols while collecting physiological data, such as EEG signals and eye-tracking information, delivering data in real time to enable precise therapy sessions. The clarity network 102 supports high-speed processing, reliable data exchange, and scalability, ensuring efficient handling of real-time patient interactions and comprehensive long-term therapeutic data management.

Further, embodiments may include a processor 104, also known as a central processing unit (CPU), which may facilitate the operation of the system according to the instructions stored in the memory 116. The processor 104 may also be a graphics processing unit (GPU). The processor 104 may include suitable logic, circuitry, interfaces, and/or code that may be configured to execute a set of instructions stored in the memory 116. The processor 104 may be a hardware component that performs arithmetic, logic, and control operations on data. The processor 104 may be comprised of the arithmetic logic unit, control unit, memory subsystems, and other subsystems. The processor 104 may be responsible for performing arithmetic and logical operations on data. The processor 104 may include components for addition, subtraction, multiplication, and division and logical operations such as AND, OR, and NOT. The processor 104 may be responsible for fetching instructions from memory 116, decoding them, and executing them. The processor 104 may manage the flow of data between different components of the system as a whole, ensuring that operations are performed in the correct order and that data is transferred efficiently. The processor 104 may provide fast access to frequently used data and instructions. The processor 104 may include components such as caches, registers, and pipelines, which are designed to minimize the time required to access and manipulate data. The processor 104 may include various other components and subsystems, such as instruction set architecture (ISA), which may define the set of instructions that the processor 104 can execute. The processor 104 may specify the format of instructions and data, the addressing modes used to access memory 116 and I/O devices, and the interrupt and exception handling mechanisms used to manage errors and other events. The processor 104 may include advanced instruction execution capabilities, support for virtualization and parallel processing, and power management mechanisms that reduce energy consumption and heat dissipation.

Further, embodiments may include a communication interface 106, which may be a hardware or software component that enables communication between two or more electronic devices or systems. The communication interface 106 may include a set of protocols, rules, and standards that define how information is transmitted and received between the devices. The communication interface 106 may be a physical connector, wireless network, or software application. It may include components such as drivers, software libraries, and firmware that may be used to control and manage the communication process. In some embodiments, the communication interface 106 may be compatible with USB, Bluetooth, or Wi-Fi. The communication interface 106 may communicate with a network. Examples of networks may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN).

Further, embodiments may include a power supply 108, which may be an electrical device or system that is used to convert electrical power from a source to a specific form or voltage that can be utilized by an electronic or electrical device. The power supply 108 may be designed to regulate and control the output power to ensure that the device or system receives the correct amount of power without any damage. The power supply 108 may include a variety of components, such as transformers, rectifiers, filters, voltage regulators, and control circuits that work together to provide the desired output voltage and current. The input power source can be from an AC or DC power source such as a battery, wall outlet, or generator. The power supply 108 may be classified based on various parameters such as the type of output voltage, power rating, efficiency, regulation, and application. In some embodiments, the power supply 108 may include various protection mechanisms such as overvoltage protection, overcurrent protection, short-circuit protection, and thermal protection to ensure safe and reliable operation.

Further, embodiments may include a response module 110, which begins with the user initiating the system. The response module 110 extracts the first VR environment from the virtual reality database 118 and the first task from the cognitive task database 122. The response module 110 sends the VR environment and cognitive task to the headset and system 128 and initiates the recording module 112. The response module 110 receives a signal or data from the mapping module 114 and determines if the signal was received from the mapping module 114. If it is determined that the signal was received from the mapping module 114, the response module 110 extracts the next VR environment from the virtual reality database 118 and the task from the cognitive task database 122, and the process returns to sending the VR environment and task to the headset and system 128. If it is determined that the signal was not received and the data was received from the mapping module 114, the response module 110 integrates the stimulation protocol data into the VR environment and cognitive task. The response module 110 sends the integrated stimulation protocol to the headset 128 and continuously polls for the completion of the stimulation protocol. The response module 110 receives a signal from the headset 128 that the stimulation protocol is completed, and the process returns to selecting the VR environment.

Further, embodiments may include a recording module 112, which begins by being initiated by the response module 110. The recording module 112 captures the performance data from the headset and system 130 and stores the received performance data in patient database 124. The recording module 112 then initiates the mapping module 114.

Further, embodiments may include a mapping module 114, which begins by being initiated by the recording module 112. The mapping module 114 extracts the patient data from the patient database 124 and analyzes the patient data. The mapping module 114 determines if the desired brain region is activated. If it is determined that the desired brain region is not activated, the mapping module 114 sends a signal to the response module 110 to select the next VR environment from the VR environment database 118 and the next task from the cognitive task database 122. If it is determined that the desired brain region is activated, the mapping module 114 extracts the corresponding stimulation protocol from the neurostimulation database 120. The mapping module 114 sends the extracted stimulation protocol data to the response module 110 and returns to the response module 110.

Further, embodiments may include a memory 116, which may store data collected by the clarity network 102, such as sensor data, analysis of data, etc. In one embodiment, the memory 116 may include suitable logic, circuitry, and/or interfaces that may be configured to store a machine code and/or a computer program with at least one code section executable by the processor 104. Examples of implementation of the memory 116 may include, but are not limited to, Random Access Memory (RAM), Read Only Memory (ROM), Hard Disk Drive (HDD), and/or a Secure Digital (SD) card.

Further, embodiments may include a virtual reality database 118, which stores, manages, and delivers immersive virtual reality scenarios and environments for therapeutic applications. The virtual reality database 118 may contain pre-configured and customizable VR content that supports a wide range of cognitive, emotional, and motor therapies. In some embodiments, the virtual reality database 118 may include scenarios such as calming natural landscapes, daily life environments, gamified tasks, cognitive challenges, and personalized content like familiar photographs or memory-based scenes that are tailored to enhance engagement and therapy effectiveness. The virtual reality database 118 contains a collection of 3D environments, animations, sounds, and interactive tasks. In some embodiments, the scenarios may be optimized for seamless integration with the headset and system 128 to ensure smooth rendering and real-time responsiveness. In some embodiments, the virtual reality database 118 may support dynamic customization, allowing the system to modify scenarios based on user-specific physiological data, such as adapting brightness, colors, difficulty levels, or the emotional valence of the content shown in response to EEG readings, pupil dilation, or task performance. For example, a cognitive task may increase in complexity as a user's engagement and neural activity improve, or calming features may be emphasized if stress indicators are detected. In some embodiments, the virtual reality database 118 may incorporate personalization capabilities, enabling users or caregivers to upload custom content, such as family photos or familiar locations, which can then be synthesized into immersive VR experiences using advanced rendering techniques. In some embodiments, the personalization feature enhances emotional connection and motivation during therapy, ultimately enhancing its efficacy and adherence. In some embodiments, the virtual reality database 118 may be integrated with the system's sensory stimulation protocols, allowing for synchronized delivery of visual and auditory stimuli within the VR environment. For example, flickering objects or modulated sounds at therapeutic frequencies can be seamlessly embedded in a gamified task or relaxation scenario. Visual and auditory stimulation may also be delivered in isolation, such as just a visual flicker and non-modulated audio, and vice versa. In some embodiments, the virtual reality database 118 may store scenarios for meditation, scenarios for relaxation, and scenarios for breathing. In some embodiments, the virtual reality database 118 may contain VR scenes, animated images, neutral or emotionally charged images or videos, sounds, music, cognitive tasks and motor tasks, and scenarios for games, and the cognitive tasks and motor tasks may require the active participation of the patient during the virtual reality immersion.

Further, embodiments may include a neurostimulation database 120, which may contain instructions for providing visual stimulation protocols, auditory stimulation protocols, and combined audio-visual stimulation protocols. The neurostimulation database 120 may store instructions for providing peripheral nerve stimulation protocols and deep brain stimulation protocols. For example, the neurostimulation database 120 may contain instructions for providing vagus nerve stimulation, transcranial direct current stimulation (tDCS), and transcranial alternating current stimulation (tACS). In some embodiments, stimulations can be provided as rhythmic stimulations. In some embodiments, the stimulation modalities may be delivered daily or according to a different schedule for a given duration. In some embodiments, the duration may be determined depending on factors like whether stimulation is active, such as provided during an assigned task, or passive, such as not provided during an assigned task, and the underlying health status of the patient. The neurostimulation database 120 may be designed to deliver targeted electrical or sensory stimulation to influence neural activity and support therapeutic outcomes. In some embodiments, the neurostimulation database 120 may contain stimulation protocols targeting specific regions of the brain to deliver personalized therapeutic interventions aimed at enhancing neural function in areas affected by neurodegenerative diseases or cognitive impairments. For example, theta-gamma stimulation protocols can target the hippocampus and medial prefrontal cortex for memory tasks. A memory task may engage a patient in a virtual maze within a VR environment, where theta-gamma entrainment is applied during the presentation of “memory tokens” or recall challenges. The stimulation synchronizes hippocampal and prefrontal cortical activity, promoting connectivity and improving memory retention. Also, gamma frequency stimulation, such as 40 Hz, may target the visual cortex to enhance visual processing or the motor cortex to improve motor coordination. For example, in a task requiring precise hand movements, gamma flicker is applied to a glowing virtual object, such as a ball, reinforcing motor cortex activity and improving the fluidity of motion. The rhythmic flicker entrains local cortical activity, helping reduce motor rigidity in conditions like Parkinson's disease. In some embodiments, for patients experiencing deficits in autobiographical memory, theta stimulation protocols may engage the Default Mode Network (DMN), which includes the medial prefrontal cortex and posterior cingulate cortex, as well as limbic structures such as the hippocampus and amygdala. A VR task may display personal photos while applying theta-frequency stimulation, directly targeting the hippocampus to support memory retrieval and leveraging the amygdala's role in emotional salience to strengthen the recall of autobiographical events. This approach encourages cross-network synchronization between the DMN and limbic system, aiding both cognitive and emotional aspects of memory retrieval. In some embodiments, beta-frequency protocols may be employed to suppress excessive beta oscillations, common in motor impairments associated with Parkinson's disease. For example, a patient performing a hand-grasping task may receive beta-reducing neurofeedback combined with gamma stimulation, targeting both motor function restoration and rigidity reduction. In some embodiments, the stimulation protocols may be dynamically adjusted based on real-time physiological data to ensure precise and effective neural modulation. In some embodiments, the neurostimulation database 120 may integrate multiple stimulation modalities, such as non-invasive brain stimulation techniques, including transcranial direct current stimulation (tDCS), transcranial alternating current stimulation (tACS), and sensory stimulations like visual and auditory entrainment. In some embodiments, the neurostimulation database 120 may modulate specific neural oscillations, such as theta, alpha, beta, gamma, etc., associated with cognitive, motor, and emotional functions, enabling treatments for conditions such as neurodegenerative diseases, cognitive decline, and motor impairments. The neurostimulation database 120 provides the delivery of stimulation protocols through electrodes, displays, and speakers embedded in the headset 130. In some embodiments, for electrical stimulation, the neurostimulation database 120 may provide low-intensity electrical currents, which are delivered via surface electrodes 136 placed on the scalp or other regions, such as peripheral nerves. These currents can be modulated at precise frequencies and amplitudes to target specific neural circuits. In some embodiments, for sensory stimulation, the neurostimulation database 120 may coordinate visual stimuli, such as flickering lights or objects displayed on the VR screen 132, and auditory stimuli, such as amplitude-modulated sound waves, at therapeutic frequencies like 4 Hz, theta, or 40 Hz, gamma, to induce neural entrainment. In some embodiments, the stimulation parameters may be adjusted dynamically based on real-time feedback from the system's sensors 136, such as EEG data, pupil dilation, or heart rate variability. For example, the system may modify the frequency, intensity, or phase of the stimulation to align with the user's current brainwave state, enhancing the therapeutic efficacy. In some embodiments, the system may also support multi-modal stimulation, combining electrical and sensory inputs to achieve synergistic effects, such as integrating tACS with synchronized visual and auditory stimuli for comprehensive neural modulation. In some embodiments, the neurostimulation database 120 may provide predefined or user-specific stimulation protocols. In some embodiments, the neurostimulation database 120 may contain safety measures, such as automatic shutdowns if abnormal neural activity, like epileptic patterns, is detected. In some embodiments, the neurostimulation database 120 also incorporates visual gratings, such as striped patterns, to induce narrow gamma oscillations in the visual cortex. The gratings, adjusted for spatial frequency, contrast, orientation, and movement, may be used to both monitor disease progression and deliver therapeutic stimulation. For example, the gamma “fingerprint” unique to an individual may serve as a sensory biomarker to track cognitive health or Alzheimer's progression. For patients with Parkinson's disease who show excessive beta activity and insufficient gamma activity in motor tasks, the neurostimulation database 120 may provide protocols for reducing rigidity and enhancing motor coordination. For example, the system may use beta power-reducing neurofeedback combined with gamma-flickering stimuli during hand movement tasks. In these tasks, patients engage in activities like squeezing a glowing, flickering ball synchronized to gamma frequencies, reinforcing motor cortex gamma oscillations, and improving the fluidity of motion. In some embodiments, multisensory convergence may be utilized by combining visual, auditory, and proprioceptive feedback to achieve coordinated motor execution. In some embodiments, the neurostimulation database 120 may integrate methods to personalize stimulation using real-time feedback from EEG and other physiological sensors. By dynamically adjusting stimulation frequencies, intensities, or phases to align with the patient's brainwave state, the system ensures therapeutic efficacy. For example, visual gratings and alternating auditory tones are synchronized to the patient's theta or gamma oscillations for tasks requiring memory recall or motor function. In some embodiments, the neurostimulation database 120 may enhance patient engagement and adherence by incorporating gamification elements. In some embodiments, tasks may reward patients for maintaining neural activity within therapeutic ranges, such as keeping a beta power bar below a threshold or focusing on a theta-flickering object. In some embodiments, the neurostimulation database 120 may support combination therapies. For example, in managing L-Dopa-induced dyskinesia and impulsivity, the system may deliver gamma protocols to reduce involuntary movements and theta protocols to improve impulse control. These therapies are personalized through calibration sessions and adjusted dynamically via AI-based algorithms informed by patient performance data.

Further, embodiments may include a cognitive task database 122, which may contain an array of cognitive tasks aimed at assessing, improving, and monitoring cognitive functions in users. The cognitive task database 122 may contain structured tasks that target areas such as memory, attention, problem-solving, emotional processing, and motor coordination. The cognitive task database 122 may provide tailored cognitive challenges that can adapt dynamically to the user's abilities and physiological responses to ensure a personalized and effective therapeutic experience. In some embodiments, the cognitive task database 122 may contain pre-designed cognitive tasks categorized by difficulty level cognitive domain, such as working memory, episodic memory, attention, etc., and therapeutic goals. In some embodiments, the tasks may be implemented in modular formats, allowing seamless integration with the VR system's sensory and feedback mechanisms. In some embodiments, the cognitive task database 122 may support both active and passive tasks, ranging from gamified challenges, such as recall and recognition games, to structured assessments, such as free recall, spatial memory mapping, pattern recognition, or affective evaluation. In some embodiments, the cognitive task database 122 may interface with the system's sensors 136 to adjust task parameters such as difficulty, pacing, and feedback in real-time based on user-specific data, including EEG signals, eye-tracking, and performance metrics. For example, a memory task could be dynamically modified to include fewer or more stimuli based on the user's accuracy and engagement levels. In some embodiments, the cognitive task database 122 may support personalized content uploads, allowing caregivers or users to incorporate familiar elements, such as family photos or known environments, into tasks to enhance emotional resonance and motivation.

Further, embodiments may include a patient database 124, which contains patient-specific information that may indicate the stimulation protocols and VR scenarios presented to given patients, and the responses of these patients to those stimulation protocols and VR scenarios. The stored responses can include physiological activity measurements as recorded by the sensors 136, health scores, and brain activity recordings. In some embodiments, the patient-specific data may also include patient-specific IDs and time stamps. In some embodiments, the patient database 124 may be accessible via a user interface of the system and/or remotely by medical teams. In some embodiments, access by medical teams may provide the teams the ability to personalize therapeutic protocols, such as the nature of an initial stimulation protocol that is to be applied for the next session of a given patient. In some embodiments, the system may also include one or more software modules that transmit, such as using encrypted transmission, the patient-specific information to a remote server or database via a computer network, allowing medical team access to the patient-specific information. In some embodiments, the patient database 124 may include the data captured by the sensors 136, such as EEG, to determine whether a specific brain region is activated during a cognitive or motor task in a VR environment. In some embodiments, the EEG sensors 136 embedded in the headset measure brainwave activity by detecting electrical signals generated by neural oscillations. These signals may be analyzed in real time to identify the frequency, amplitude, and spatial distribution of brain activity associated with specific neural circuits. For example, gamma oscillations, such as 30-100 Hz, measured over the occipito-parietal regions may indicate activity in the visual cortex during visual memory or spatial navigation tasks. Similarly, theta rhythms, such as 4-8 Hz, originating from the medial prefrontal cortex, the precuneus or hippocampus suggest activation of the Default Mode Network during memory recall or self-referential thinking tasks. Likewise, theta rhythms measured over the medial prefrontal cortex (mPFC) may indicate activation of the limbic system, including structures such as the hippocampus and amygdala, during the observation of emotionally charged flickered content. In some embodiments, other sensor 136 data, such as event-related potentials or ERPs, may be extracted to determine task-specific responses, such as the P300 waveform associated with decision-making and attention or the LPP (Late Positive Potential) linked to the processing of emotionally salient stimuli. Spatial localization algorithms, such as source reconstruction, use the EEG signal to map the activity to specific brain regions. If the EEG data indicates insufficient neural activation in the targeted area, such as weak gamma power in the visual cortex during a visuospatial task, the system adjusts the stimulation protocol or selects alternative tasks to better engage the desired brain region. In some embodiments, additional sensors 136, such as eye-tracking data, may complement EEG analysis by confirming the patient's focus on stimuli relevant to the task. For example, fixation on a flickering object paired with EEG-detected gamma power increase confirms engagement of the visual cortex.

Further, embodiments may include a cloud 126, which may be a network of remote servers that provide on-demand computing resources and services over the Internet. The cloud 126 may consist of a collection of servers, storage devices, and networking equipment. Users may access the cloud 126 through a variety of devices, such as computers, smartphones, and tablets, using internet connectivity. In some embodiments, the architecture of the cloud 126 may be based on a distributed computing model, with multiple servers working together to provide services to users.

Further, embodiments may include a headset and system 128, which may be a wearable device designed to deliver therapeutic interventions through immersive virtual environments and sensory stimulation. The headset and system 128 may feature high-resolution, high-refresh-rate, such as 120 Hz or higher, LCD or OLED displays, ensuring crisp visuals and the capability to present flickering stimuli at specific frequencies, such as 40 Hz for gamma brainwave entrainment. The headset and system 128 may be integrated with advanced sensors 136 that measure a variety of physiological parameters, including brain activity via electroencephalogram (EEG) sensors, pupil dilation using pupillometry, and eye-tracking to monitor gaze direction and detect whether the eyes are open or closed. In some embodiments, additional sensors may be integrated to measure heart rate, variability, and skin conductance, to provide data to assess stress and engagement levels. In some embodiments, the headset and system 128 may include stereo or spatial audio speakers 132 that deliver synchronized auditory stimuli at therapeutic frequencies, complementing the visual inputs for multisensory entrainment. The headset and system 128 may be ergonomically designed with an adjustable, padded headband for comfortable extended use and feature a lightweight build to reduce fatigue. In some embodiments, an RGB LED indicator light may provide visual feedback on the device status, such as activation, pairing mode, or alerts, including medical emergencies. In some embodiments, power may be supplied via a rechargeable battery with optional direct plug-in or cradle charging support, enabling uninterrupted operation during sessions. In some embodiments, the headset and system 128 may communicate with the clarity network 102, including the virtual reality database 118, neurostimulation database 120, and cognitive task database 122. The databases may include pre-configured visual and auditory stimulation patterns and immersive environments like cognitive games or calming landscapes tailored to enhance engagement and therapeutic outcomes. In some embodiments, the headset and system 128 may communicate with the response module 110 and record module 112, which may analyze real-time physiological responses, dynamically adjusting stimulation frequencies and amplitudes to optimize neural entrainment. In some embodiments, the system may support remote monitoring and control, enabling caregivers and medical professionals to access patient progress and physiological data securely and make adjustments as needed. In some embodiments, the frequencies at which sensory stimulations are provided may include, for example, gamma-corresponding frequencies in the 30-100 Hz range. In some embodiments, it may be required that the screen 130 of the headset and system 128 have an appropriately high enough refresh rate. In some embodiments, it may be required that the screen 130 have a refresh rate that is an integer multiple of those stimulation frequencies that are to be presented. For example, where the stimulation frequencies that are to be presented include a 40 Hz visual stimulus, given the Nyquist rule, 80 frames per second can be a minimum refresh rate for the screen of the headset and system 128. In some embodiments, a headset screen 130 refresh rate of 120 Hz or higher may be used. For example, a headset screen 130 refresh rate of 160 Hz, 200 Hz, or 240 Hz can provide for a stable delivery of 40 Hz visual stimulation.

Further, embodiments may include a screen 130, which may be a high-performance display panel embedded within the headset that is responsible for presenting immersive virtual environments and delivering precise visual stimuli for therapeutic interventions. In some embodiments, the screen 130 may be remotely monitored, either at intervals or continuously, to ensure stimulation stability in order to dynamically adapt the rendering, frames per second, or battery expenditure, etc., in order to prioritise flicker stability. The screen 130 may be designed to support dynamic content delivery with high refresh rates, resolution, and brightness, enabling both passive and active engagement with the virtual reality scenarios and sensory stimulation protocols. In some embodiments, the screen 130 may be a high-definition, HD, ultra-high-definition, UHD, LCD, or OLED display optimized for therapeutic use. In some embodiments, the screen 130 may feature refresh rates of 120 Hz or higher to accurately present flickering visual stimuli at target frequencies, such as 40 Hz for gamma brainwave entrainment, while maintaining visual stability and avoiding latency. The resolution, such as 4K or higher, may provide visuals that are sharp, reducing eye strain and improving immersion. In some embodiments, the screen's 130 brightness and color accuracy may be adjustable to support a wide range of therapeutic scenarios, from calming, low-light environments to more vivid, high-contrast tasks. In some embodiments, the screen 130 may deliver full-environment modulation, where the entire display flickers at a specific frequency or localized stimulation through modulated objects or frames within the VR environment. In some embodiments, advanced rendering techniques may allow sinusoidal or square-wave luminance modulation for precise visual flicker delivery. In some embodiments, the system's ability to synchronize visual stimuli with auditory inputs further enhances multisensory neural entrainment, with phase-locking functionality ensuring alignment between visual and auditory frequencies. In some embodiments, the screen 130 may interface with the VR headset's sensors 136, including eye-tracking and pupillometry, to monitor user engagement and focus. For example, the screen 130 may adapt stimuli based on real-time physiological feedback, such as EEG data or gaze patterns, to optimize therapeutic efficacy. In some embodiments, the screen 130 refresh rate and modulation patterns may be precisely controlled to minimize visual artifacts, such as flickering instability. In some embodiments, integrated software may dynamically adjust screen 130 parameters, such as frequency, brightness, and color intensity, in response to patient-specific requirements or feedback loops. In some embodiments, the screen 130 may be used for displaying active cognitive and functional tasks. In some embodiments, the screen 130 may display gamified tasks, visual memory tests, or familiar environments, such as personalized photos or landscapes, making the therapy more engaging and personalized.

Further, embodiments may include speakers 132, which may be integrated audio output devices that provide high-quality sound, including therapeutic auditory stimuli, as part of the sensory stimulation protocols. In some embodiments, the speakers 132 may be positioned near the user's ears and may be designed to produce sound with a wide frequency range to provide clarity and precision across various auditory inputs. In some embodiments, the speakers 132 may be optimized for therapeutic purposes, featuring low distortion and high fidelity to preserve the integrity of the auditory signals. The speakers 132 may utilize bone conduction. In some embodiments, the system may dynamically synchronize the auditory and visual stimuli to maintain precise phase alignment to enhance neural entrainment and therapeutic efficacy. For example, auditory stimuli at 40 Hz can be phase-locked with visual flickers at the same frequency or complementary frequencies, allowing the system to target specific neural pathways effectively. In some embodiments, the speakers 132 may provide task-related audio guidance and feedback, such as instructions, questions, or results during cognitive and functional activities. In some embodiments, the speakers 132 may create immersive soundscapes by reproducing environmental sounds, music, or personalized audio elements to complement the virtual reality environment. In some embodiments, the system's processing modules may manage the audio delivery, adapting sound patterns in real time based on physiological feedback, such as EEG data or pupil dilation.

Further, embodiments may include electrodes 134, which may be specialized components designed to monitor and, in some cases, modulate the user's neural and physiological activity. The electrodes 134 may be used to collect real-time data on brain activity via EEG, enabling the system to analyze neural oscillations, such as theta, alpha, beta, and gamma brainwaves, to tailor therapeutic interventions. In some embodiments, the electrodes 134 may be non-invasive and embedded within the headset 128 to maintain user comfort during extended sessions. In some embodiments, the electrodes 134 may be positioned to ensure optimal contact with the scalp, typically around the frontal, parietal, central, or occipital regions, depending on the specific neural signals being targeted. In some embodiments, the electrodes 134 may support peripheral nerve stimulation or deliver electrical signals for neuromodulation, such as transcranial direct current stimulation (tDCS) or transcranial alternating current stimulation (tACS), providing advanced therapeutic capabilities. In some embodiments, the electrodes 134 may be designed to minimize noise and maximize signal fidelity, employing high-precision amplification and filtering techniques to capture subtle electrical signals from the brain. In some embodiments, the collected data may include brainwave phase, amplitude, and frequency, which are processed in real time by the system's computational modules. In some embodiments, the information allows the system to adjust sensory stimulation protocols dynamically, such as aligning visual or auditory stimuli with the user's neural activity to optimize entrainment and therapeutic outcomes. In some embodiments, the electrodes 134 may deliver low-level electrical currents, modulated at specific frequencies, to target neural pathways associated with cognitive, affective, or motor functions. In some embodiments, the electrodes 134 may contribute to monitoring stress or abnormal neural activity, such as epileptic patterns. They may trigger safety mechanisms like stopping stimulation or alerting caregivers in critical situations.

Further, embodiments may include sensors 136, which may be designed to monitor a wide range of physiological and behavioral metrics, providing real-time data that enhances the therapeutic effectiveness of the system. In some embodiments, the sensors 136 may include electroencephalogram (EEG) sensors to measure brain activity, pupillometry sensors to track pupil dilation, eye-tracking sensors to monitor gaze direction and eye openness, and heart rate sensors to evaluate cardiac activity and variability. In some embodiments, additional sensors 136 may measure skin conductance, respiratory rate, body temperature, and muscle activity via electromyography (EMG). In some embodiments, the EEG sensors 136 may be positioned to capture brainwave activity across different frequency bands, such as theta, alpha, beta, and gamma, which are associated with various cognitive and motor functions. In some embodiments, the EEG sensors 136 embedded in the headset measure brainwave activity by detecting electrical signals generated by neural oscillations. These signals may be analyzed in real-time to identify the frequency, amplitude, and spatial distribution of brain activity associated with specific neural circuits. For example, gamma oscillations, such as 30-100 Hz, measured over the occipito-parietal regions may indicate activity in the visual cortex during visual memory or spatial navigation tasks. Similarly, theta rhythms, such as 4-8 Hz, originating from the medial prefrontal cortex or hippocampus suggest activation of the Default Mode Network during memory recall or self-referential thinking tasks. In some embodiments, other sensor 136 data, such as event-related potentials or ERPs, may be extracted to determine task-specific responses, such as the P300 waveform associated with decision-making and attention or the LPP (Late Positive Potential) linked to the processing of emotionally salient stimuli. Spatial localization algorithms, such as source reconstruction, use the EEG signal to map the activity to specific brain regions. If the EEG data indicates insufficient neural activation in the targeted area, such as weak gamma power in the visual cortex during a visuospatial task, the system adjusts the stimulation protocol or selects alternative tasks to better engage the desired brain region. In some embodiments, additional sensors 136, such as eye-tracking data, may complement EEG analysis by confirming the patient's focus on stimuli relevant to the task. For example, fixation on a flickering object paired with EEG-detected gamma power increase confirms engagement of the visual cortex. In some embodiments, the pupillometry and eye-tracking sensors 136 may work in tandem to assess focus and attention by detecting gaze patterns and pupil responses to visual stimuli. In some embodiments, heart rate sensors 136 may provide insights into stress and relaxation states, while skin conductance sensors 136 may measure arousal levels through changes in sweat gland activity. In some embodiments, the data from the sensors 136 may be processed in real time by the system's computational modules, which analyze physiological responses and adjust the sensory stimulation protocols accordingly. For example, EEG signals may guide the system to modify visual or auditory stimuli frequencies to align with the user's brainwave phase, optimizing neural entrainment. Also, eye-tracking data may prompt adjustments in visual stimuli placement, salience or valence to ensure the user remains engaged. In some embodiments, the sensors 136 may serve safety functions, such as detecting abnormal neural activity or stress states, triggering alerts to caregivers, or halting therapy when necessary.

Further, embodiments may include a communication interface 138, which may be a hardware or software component that enables communication between two or more electronic devices or systems. The communication interface 138 may include a set of protocols, rules, and standards that define how information is transmitted and received between the devices. The communication interface 138 may be a physical connector, wireless network, or software application. It may include components such as drivers, software libraries, and firmware that may be used to control and manage the communication process. In some embodiments, the communication interface 138 may be compatible with USB, Bluetooth, or Wi-Fi. The communication interface 138 may communicate with a network. Examples of networks may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN).

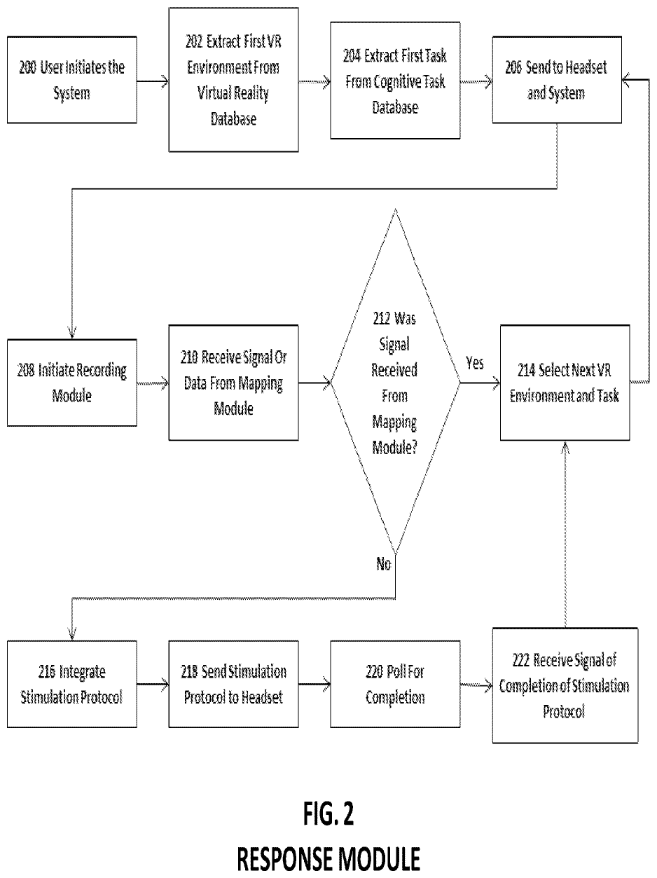

FIG. 2 illustrates the response module 110. The process begins with the user initiating, at step 200, the system. In some embodiments, the response module 110 may be activated by the user or through inputs made on the headset 128 interface. In some embodiments, the headset 128 may connect to the response module 110, allowing the user to select session parameters such as mode, such as a testing mode or stimulation mode. The selections may be transmitted to the response module 110, which configures the session accordingly. In some embodiments, the response module 110 may retrieve the necessary configuration settings, including database pointers for the virtual reality database 118, cognitive task database 122, and neurostimulation database 120 to ensure they are ready for direct access. In some embodiments, the response module 110 may also verify that the headset 128 sensors 136, such as EEG sensors, heart rate monitors, and eye-tracking devices, are functional and capable of collecting data during the session. In some embodiments, the user may manually select a desired brain region to target specific cognitive or motor deficits. In some embodiments, the system may autonomously select the desired brain region based on predefined algorithms that analyze patient-specific parameters, therapeutic goals, or historical performance trends. In some embodiments, a medical professional may select the desired brain region, leveraging their clinical expertise to tailor the therapy based on the patient's condition and treatment progress. In some embodiments, the system may retrieve historical patient data stored in the patient database 124, such as previously targeted brain regions, engagement scores, or therapy outcomes, to either continue previous therapy sessions or adapt the stimulation protocols to advance the patient's therapeutic progress.

The response module 110 extracts, at step 202, the first VR environment from the virtual reality database 118. The response module 110 retrieves the first VR environment from the virtual reality database 118. In some embodiments, the VR environment may be selected based on its alignment with therapeutic testing, patient-specific requirements, or targeting a specific brain region of the patient. In some embodiments, parameters such as environmental visuals, audio settings, and immersive complexity are loaded for integration with the upcoming cognitive task. In some embodiments, the selection may be informed by patient history or pre-defined session configurations. The response module 110 extracts, at step 204, the first task from the cognitive task database 122. The response module 110 retrieves the first cognitive task from the cognitive task database 122. In some embodiments, the task may be designed to test specific cognitive domains, such as memory, attention, emotional processing or problem-solving, that may be correlated to a specific brain region. In some embodiments, the cognitive task parameters, such as difficulty level and interaction design, may be aligned with the selected VR environment to create a cohesive and engaging experience to ensure the tasks are appropriately challenging for the user's therapeutic and testing needs.

The response module 110 sends, at step 206, the VR environment and cognitive task to the headset and system 128. The selected VR environment and cognitive task are transmitted to the headset 128 for display and interaction through the cloud 126 via the communication interface 106. For example, the system may implement the Montreal Cognitive Assessment (MoCA) and Unified Parkinson's Disease Rating Scale (UPDRS) in a virtual reality environment to evaluate cognitive and motor impairments in an immersive and interactive manner. The testing in the virtual reality environment may enhance patient engagement, enable precise data collection, and integrate real-time monitoring to tailor therapeutic interventions. In some embodiments, the MoCA is a tool used to detect mild cognitive impairments. It is divided into its core domains, such as visuospatial abilities, memory, attention, language, and executive functioning, which are recreated as interactive VR tasks. For example, visuospatial tasks may involve navigating through a 3D maze to locate landmarks or solve spatial puzzles, during which gamma oscillations in the hippocampus and prefrontal cortex are monitored via sensors 136 embedded in the headset 128. In some embodiments, the UPDRS assesses both motor and non-motor symptoms of Parkinson's disease and is incorporated into the VR environment. In some embodiments, motor tasks, such as repetitive hand movements or object manipulation, may be performed with real-time feedback mechanisms, allowing the system to measure key parameters like speed, amplitude, and fluidity of motion. In some embodiments, multisensory stimuli, including flickering visual cues and auditory tones, may be integrated to induce therapeutic neural oscillations, such as gamma and theta waves, and to dynamically adjust task difficulty based on patient performance. In some embodiments, the system may initiate real-time monitoring of the user's physiological responses, such as EEG activity, heart rate, and eye tracking. In some embodiments, the environment and task may be designed to engage the user while providing meaningful data on cognitive performance and neural activity. In some embodiments, the response module 110 may begin collecting physiological parameters, such as brainwave frequencies, pupillometry data, and motor responses, to ensure the VR environment and tasks align with patient-specific treatment tests. In some embodiments, the data points may be used to refine ongoing and future stimulation protocols by analyzing neural oscillation patterns and engagement levels.

The response module 110 initiates, at step 208, the recording module 112. The recording module 112 begins by being initiated by the response module 110. The recording module 112 captures the performance data from the headset and system 130 and stores the received performance data in patient database 124. The recording module 112 analyzes the patient data and extracts the stimulation protocol from the neurostimulation database 120. The recording module 112 sends the stimulation protocol to the mapping module 114 and initiates the mapping module 114.

The response module 110 receives, at step 210, a signal or data from the mapping module 114. If the response module 110 receives a signal, the response module 110 interprets it as an indication to proceed with selecting the next VR environment and cognitive task. In some embodiments, the signal may signify that the desired brain region has not been sufficiently activated, prompting the response module 110 to retrieve new therapeutic content from the virtual reality database and cognitive task database, thereby advancing the therapy session. For example, if the mapping module 114 determines that the desired brain region is not sufficiently activated during a spatial navigation task, it sends a signal to the response module to select a new VR environment and task, such as a visuospatial puzzle or memory maze, to better engage the target region. If the response module 110 receives data instead, it may include a stimulation protocol extracted by the mapping module 114 from the neurostimulation database 120. The response module 110 integrates the received stimulation protocol into the ongoing VR environment and cognitive task, configuring the combined content for delivery to the headset. For example, if the mapping module 114 confirms that the desired brain region, such as the medial prefrontal cortex, is activated during a decision-making task, it may send data for a theta-gamma stimulation protocol to the response module 110. The response module 110 then integrates this stimulation protocol into the VR environment and cognitive task, such as highlighting specific decision paths with flickering visual cues synchronized to therapeutic frequencies.

The response module 110 determines, at step 212, if the signal was received from the mapping module 114. The response module 110 may determine the next step in the process by determining if a signal was received or if a data packet was received from the mapping module 114. For example, a signal functions as a simple directive for the response module 110 to proceed with task and environment selection. If the mapping module 114 identifies insufficient activation in a target brain region, such as the hippocampus, during a memory task, it sends a signal prompting the response module 110 to retrieve a new task and VR environment from the respective databases. In some embodiments, the new selections may involve transitioning to a different cognitive task, like a sequence recall game or emotional rating of specific images, to better engage the hippocampus. A data packet contains more detailed information, including a specific stimulation protocol extracted from the neurostimulation database 120. For example, if the mapping module 114 confirms activation of the targeted brain region, such as the prefrontal cortex, it sends a data packet detailing a theta-gamma stimulation protocol. The response module 110 integrates this protocol into the current VR environment and cognitive tasks, such as overlaying flickering visual stimuli on decision-making cues within the VR scene.

If it is determined that the signal was received from the mapping module 114, the response module 110 extracts, at step 214, the next VR environment from the virtual reality database 118 and task from the cognitive task database 122, and the process returns to sending the VR environment and task to the headset and system 128. If a signal is received, the response module 110 is prompted to extract the next VR environment from the virtual reality database 118 and the next task from the cognitive task database 122. The response module 110 retrieves the VR environment and task to ensure that their complexity aligns with the patient's current performance metrics as recorded in the patient database 124. For example, if the patient has completed a visuospatial navigation task successfully, the next task might introduce a slightly more complex spatial puzzle. The newly selected VR environment and task are then transmitted to the headset 128 for interaction.

If it is determined that the signal was not received and the data was received from the mapping module 114, the response module 110 integrates, at step 216, the stimulation protocol data into the VR environment and cognitive task. In some embodiments, the integration process begins by aligning the received stimulation protocol, such as visual flickering and auditory cues, both etc. with the selected VR environment and cognitive task to ensure a cohesive therapeutic session where neurostimulation parameters enhance the efficacy of the task without disrupting its flow. For example, if the stimulation protocol specifies a 40 Hz gamma-frequency visual flicker to target neural oscillations in the prefrontal cortex, the response module 110 incorporates this flickering effect into a task where the user selects the most emotionally significant image from a set, requiring active decision-making and emotional evaluation. In another example, this flickering effect could be integrated by the response module 110 into dynamic elements of the VR environment, such as a flickering object in a visually engaging scene, which may include a glowing geometrical shape like a circle or pulsating light source. In some embodiments, auditory stimulation at complementary frequencies may be embedded into the environment, such as alternating tones or rhythmic beats that match the visual flicker, creating a synchronized multi-sensory experience. In some embodiments, the integration may also incorporate advanced visual stimulation methods, such as gratings or dynamic textures. For example, in a cognitive task requiring attention and focus, objects in the VR environment may display alternating striped patterns or changing contrast levels to deliver therapeutic stimulation. In some embodiments, the gratings may adjust dynamically, varying spatial frequency, orientation, or movement, to fine-tune the engagement of specific brain regions like the visual cortex or default mode network. In some embodiments, the system may incorporate modulated soundscapes that align with the task's rhythm for auditory components, such as a steady tone transitioning in amplitude or pitch.

The response module 110 sends, at step 218, the integrated stimulation protocol to the headset 128. The response module 110 transmits the integrated stimulation protocol, combined with the selected VR environment and cognitive task, to the headset 128. In some embodiments, the transmission may involve packaging the protocol's visual, auditory, or multi-sensory components into a format compatible with the headset's 128 screen 130 and sensor 136 systems. In some embodiments, the stimulation parameters, such as visual flickering frequencies or auditory tones, may be synchronized with the VR environment's content to ensure a seamless therapeutic session. For example, if the protocol involves a 40 Hz flickering object in a VR scene, the response module 110 ensures that the object's flickering effect is precisely aligned with the scene's spatial and temporal dynamics.

The response module 110 continuously polls at step 220 for the completion of the stimulation protocol. The response module continuously monitors the stimulation protocol's execution within the headset 128 by polling the system. In some embodiments, polling involves sending periodic queries to the headset 128 to retrieve status updates on the stimulation session's progress. In some embodiments, the headset's 128 sensors 136 and processing systems provide real-time feedback, such as the current status of the visual flicker, auditory tones, and physiological data being recorded. For example, the response module 110 may verify whether the flickering object in the VR environment is being displayed at the correct frequency or whether the auditory tones are being played as specified in the stimulation protocol. The response module 110 receives, at step 222, a signal from the headset 128 that the stimulation protocol is completed, and the process returns to selecting the VR environment. In some embodiments, the signal may be generated by the headset's 128 software after all components of the protocol, such as visual flickers, auditory cues, or specific cognitive tasks, have been successfully delivered and the session duration has elapsed. The response module 110 confirms the completion signal and updates the session's status in the system, recording it as completed. For example, if the protocol involved 40 Hz visual flicker for a pre-set duration, the headset 128 would confirm that the flicker was executed continuously and uninterrupted for the specified time. Once the signal is received, the response module 110 transitions the system back to selecting the next VR environment and cognitive task.

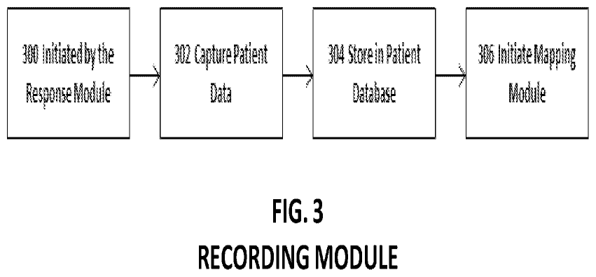

FIG. 3 illustrates the recording module 112. The process begins with the recording module 112 being initiated at step 300 by the response module 110. In some embodiments, the recording module 112 may be initiated simultaneously with the response module 110. In some embodiments, the recording module 112 may connect to the headset 128 through the cloud 126 via the communication interface 106. The recording module 112 captures, at step 302, the performance data from the headset and system 128. The headset 128 sends real-time performance data to the recording module 112, including physiological signals, such as EEG data reflecting brainwave activity, heart rate variability, etc., and cognitive task metrics, such as accuracy, response time, etc. In some embodiments, the system may analyze the collected data to identify key trends and variations in the patient's physiological responses. In some embodiments, the recording module 112 may evaluate the nature and variation of the physiological data to calculate health scores, such as global health or entrainment scores, that indicate the patient's progress and alignment with desired therapeutic outcomes. In some embodiments, the recording module 112 may also detect abnormal neural activity patterns, such as epileptiform discharges, halting the task, or adjusting the VR environment to prioritize safety. The recording module 112 may also capture eye-tracking data to assess patient engagement. In some embodiments, the data may include data from the EEG sensors 136 embedded in the headset 130 that measure brainwave activity by detecting electrical signals generated by neural oscillations. These signals may be analyzed in real-time to identify the frequency, amplitude, and spatial distribution of brain activity associated with specific neural circuits. For example, gamma oscillations, such as 30-100 Hz, measured over the occipito-parietal regions may indicate activity in the visual cortex during visual memory or spatial navigation tasks. Similarly, theta rhythms, such as 4-8 Hz, originating from the medial prefrontal cortex or hippocampus suggest activation of the Default Mode Network during memory recall or self-referential thinking tasks. In some embodiments, other sensor 136 data, such as event-related potentials or ERPs, may be extracted to determine task-specific responses, such as the P300 waveform associated with decision-making and attention or the LPP (Late Positive Potential) linked to the processing of emotionally salient stimuli. Spatial localization algorithms, such as source reconstruction, use the EEG signal to map the activity to specific brain regions. If the EEG data indicates insufficient neural activation in the targeted area, such as weak gamma power in the visual cortex during a visuospatial task, the system adjusts the stimulation protocol or selects alternative tasks to better engage the desired brain region. In some embodiments, additional sensors 136 data, such as eye-tracking data, may complement EEG analysis by confirming the patient's focus on stimuli relevant to the task. For example, fixation on a flickering object paired with EEG-detected gamma power increase confirms engagement of the visual cortex.

The recording module 112 stores, at step 304, the received performance data in patient database 124. The collected performance and physiological data are stored in the patient database 124 for detailed analysis. In some embodiments, data cleaning algorithms may be employed to filter out erroneous or outlier data points to ensure the stored dataset accurately reflects the user's performance. In some embodiments, the stored data may be used to track the evolution of parameters within the session, compare progress across multiple sessions to assess long-term treatment efficacy, and inform further detailed analysis.

The recording module 112 initiates, at step 306, the mapping module 114. The mapping module 114 begins by being initiated by the recording module 112. The mapping module 114 extracts the patient data from the patient database 124 and analyzes the patient data. The mapping module 114 determines if the desired brain region is activated. If it is determined that the desired brain region is not activated, the mapping module 114 sends a signal to the response module 110 to select the next VR environment from the VR environment database 118 and the next task from the cognitive task database 122. If it is determined that the desired brain region is activated, the mapping module 114 extracts the corresponding stimulation protocol from the neurostimulation database 120. The mapping module 114 sends the extracted stimulation protocol data to the response module 110 and returns to the response module 110.

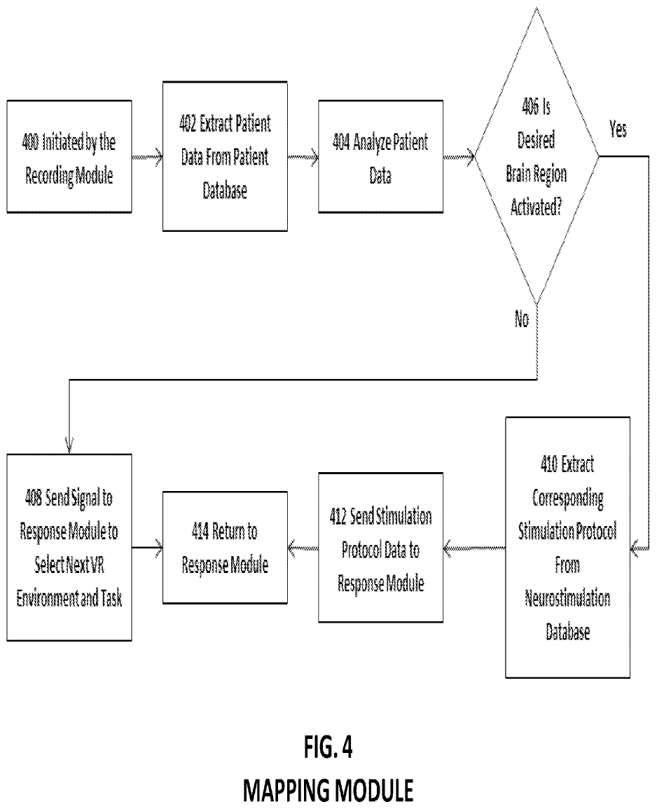

FIG. 4 illustrates the mapping module 114. The process begins with the mapping module 114 being initiated at step 400 by the recording module 112. The mapping module 114 extracts, at step 402, the patient data from the patient database 124. The patient data may include physiological responses, such as EEG readings, heart rate variability, eye-tracking data, etc., task performance metrics, such as accuracy, reaction times, etc., and historical therapy records. For example, EEG data might include oscillatory patterns like theta or gamma rhythms, while performance data could highlight success rates in cognitive tasks.

The mapping module 114 analyzes, at step 404, the patient data, including the desired brain region that was determined by the user, a medical professional, historical patient data stored in the patient database 124, or automated system configurations. The mapping module 114 analyzes the extracted patient data to identify patterns, trends, and metrics relevant to the therapy session. The analysis may involve examining EEG signals for specific neural oscillations, such as gamma activity in the occipital lobe, correlating task performance data with brain activity, and comparing physiological responses to expected outcomes. For example, if the session targeted memory recall, the mapping module 114 may evaluate the presence of theta-gamma coupling in the hippocampus and prefrontal cortex, as well as task accuracy, to assess the effectiveness of the VR environment and cognitive task. In some embodiments, the mapping module 114 may analyze the patient data, specifically EEG signals, to assess whether the desired brain region is activated during the VR task. In some embodiments, the process may involve the examination of specific neural oscillations, amplitudes, and frequencies associated with the target brain region. For example, gamma activity, such as 30-100 Hz, in the hippocampus is often linked to memory tasks, while theta activity, such as y 4-8 Hz, is commonly observed in the prefrontal cortex during tasks requiring attention and working memory. In some embodiments, the mapping module 114 compares the recorded EEG data against predefined thresholds stored in the neurostimulation database 120. In some embodiments, the thresholds may be determined from clinical studies and patient-specific baselines. For example, during a memory recall task, the system monitors gamma power in the 40 Hz range. If the EEG data shows a power increase exceeding 10% above baseline in the hippocampal or the posterior parietal cortex region, it indicates activation. If gamma power is below the threshold, it suggests suboptimal engagement. For tasks like decision-making, theta power is analyzed. A target threshold may be a 15% increase in theta amplitude in the prefrontal cortex relative to the baseline. If the increase falls short of this threshold, the system concludes that the desired activation has not occurred. In some embodiments, the analysis may also consider spatial patterns using EEG sensors 136 placed to capture signals from specific brain areas. For example, a high-density electrode array ensures precise measurement of occipito-parietal gamma oscillations for visuospatial tasks. In some embodiments, the mapping module 114 may account for noise and artifacts in the data, such as those from eye blinks or muscle movements, by employing data-cleaning algorithms. Once clean EEG data is obtained, power spectral density and coherence analyses help confirm whether the recorded brain activity aligns with the target region's expected response. In some embodiments, if the thresholds are not met, the mapping module 114 may determine that the desired brain region is not sufficiently activated and sends a signal to the response module 110. If the thresholds are met, the system validates that the desired brain region is engaged, advancing to extracting a corresponding stimulation protocol.

The mapping module 114 determines, at step 406, if the desired brain region is activated. The mapping module 114 evaluates whether the desired brain region has been successfully activated based on the analyzed patient data. In some embodiments, activation may be determined using pre-defined thresholds for neural activity or physiological responses. For example, if the target was the hippocampus, the mapping module 114 may assess whether theta rhythms exceeded a specific power level during a memory task. If the thresholds are met, the mapping module 114 may conclude that the desired brain region is activated, and if not, the mapping module 114 may identify the need for adjustments.

If it is determined that the desired brain region is not activated, the mapping module 114 sends, at step 408, a signal to the response module 110 to select the next VR environment from the VR environment database 118 and the next task from the cognitive task database 122. In some embodiments, the signal may instruct the response module 110 to select a different VR environment and cognitive task from the VR environment database 118 and cognitive task database 122, respectively. For example, if the current environment and task fail to engage the hippocampus, the system may switch to a more immersive memory task or adjust the VR scenario to better align with the patient's therapeutic needs.

If it is determined that the desired brain region is activated, the mapping module 114 extracts, at step 410, the corresponding stimulation protocol from the neurostimulation database 120. In some embodiments, the stimulation protocol may be selected based on the patient's physiological data, task performance, and the therapeutic goals of the session. For example, if the hippocampus is activated during a memory task, the system might select a 40 Hz gamma stimulation protocol to enhance neural activity and strengthen connectivity in that region. In some embodiments, the process may involve matching the activation patterns observed in the EEG data with predefined stimulation protocols optimized for the desired brain region and cognitive task. Using the results of the mapping module's 114 analysis the mapping module 114 may identify the brain region activated and the degree of activation. For example, if the hippocampus shows suboptimal gamma oscillations, such as less than the required 40 Hz power increase threshold, the system selects a gamma stimulation protocol tailored to enhance activity in this region. If the prefrontal cortex exhibits weak theta oscillations during a working memory task, the mapping module 114 may extract a theta stimulation protocol designed to amplify connectivity within the relevant neural network. In some embodiments, the extracted stimulation protocol may specify key parameters, including the stimulation frequency, intensity, and modality. For example, a stimulation protocol designed for gamma stimulation to enhance memory tasks might deliver 40 Hz visual flickering via the VR display, paired with auditory cues at the same frequency, to reinforce hippocampal activity. Similarly, a stimulation protocol targeting theta stimulation for working memory tasks could involve slow visual gratings oscillating at 6 Hz, combined with rhythmic auditory tones, to enhance theta synchronization in the prefrontal cortex. In some embodiments, the neurostimulation database 120 may organize stimulation protocols by brain region, task type, and stimulation modality. For example, a protocol for visuospatial memory tasks may combine narrow gamma visual gratings in the occipito-parietal region with tactile feedback to engage motor and sensory cortices. For autobiographical memory tasks, a theta-gamma coupling protocol may pair familiar visual stimuli, such as family photos, with alternating theta-gamma audio patterns, targeting the default mode network. In some embodiments, the mapping module 114 may tailor the stimulation protocol to individual patient characteristics stored in the patient database. For example, if previous sessions indicate that a patient responds best to 38 Hz gamma stimulation rather than the typical 40 Hz, the system selects a protocol with this specific frequency. If the patient's EEG shows excessive noise in certain channels, the mapping module 114 may prioritize auditory over visual stimuli to ensure effective entrainment. During a memory maze task, the mapping module 114 may identify suboptimal hippocampal activation due to low gamma power, such as 5% below the desired threshold. The extracted stimulation protocol provides 40 Hz flickering in the VR environment's maze walls, synchronized with subtle auditory chimes. In some embodiments, the targeted stimulation may be designed to enhance neural synchronization and improve memory recall.

The mapping module 114 sends, at step 412, the extracted stimulation protocol data to the response module 110. In some embodiments, the stimulation protocol data may include the parameters for delivering the stimulation, such as frequency, intensity, and modality, such as visual flickering or auditory tones. For example, a 40 Hz gamma stimulation protocol targeting the visual cortex might specify flickering parameters to synchronize with the patient's neural activity. The response module 110 receives the data and integrates the protocol into the ongoing VR environment and cognitive task. The mapping module 114 returns, at step 414, to the response module 110.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

What is claimed is:

1. A method for biomarker-guided personalized neuromodulation therapy delivered within a virtual-reality environment, the method comprising:
storing in memory baseline data of biomarkers associated with one or more neurological conditions;
receiving, via a communication interface, biomarker data associated with a user from one or more external clinical systems;
extracting one or more features from the biomarker data for one or more deviations from the baseline data;
scoring the features based on the deviations, wherein the score is weighted based on indicators of significance;
determining one or more therapeutic targets based on the scored features; and
providing a neuromodulation protocol in the virtual-reality environment based on the determined therapeutic targets.

2. The method of claim 1, further comprising receiving the baseline data from the one or more external clinical systems via the communication interface, wherein the baseline data includes electrophysicological signals.

3. The method of claim 1, further comprising receiving sensor data from a virtual-reality headset.

4. The method of claim 1, wherein scoring the features includes assigning a higher weight to specific features characteristic of a particular pathological process.

5. The method of claim 1, further comprising generating a biomarker profile based on the scored biomarker features.

6. The method of claim 1, wherein providing the neuromodulation protocol includes selecting one or more virtual-reality environment assets.

7. The method of claim 1, wherein providing the neuromodulation protocol includes selecting one or more neuromodulation parameters based on the determined therapeutic targets configured to modulate the user.

8. The method of claim 1, further comprising updating the neuromodulation protocol based on an analysis of a user response to the provided protocol, wherein updating the neuromodulation protocol includes adjusting one or more neuromodulation parameters.

9. The method of claim 8, wherein neuromodulation parameters includes a temporal frequency of visual and auditory stimulation.

10. The method of claim 1, further comprising tracking the scored features over time to predict a disease progression.

11. A system for biomarker-guided personalized neuromodulation therapy delivered within a virtual-reality environment, the system comprising:
memory that stores baseline data of biomarkers associated with one or more neurological conditions; and
a processor that executes instructions in memory, wherein the processor executes instructions to:
receive, via a communication interface, biomarker data associated with a user from one or more external clinical systems;
extract one or more features from the biomarker data for one or more deviations from the baseline data;
score the biomarker features based on the deviations, wherein the score is weighted based on indicators of significance;
determine one or more therapeutic targets based on the scored biomarker features; and
provide a neuromodulation protocol in the virtual-reality environment based on the determined therapeutic targets.

12. The system of claim 11, wherein the processor executes further instructions to receive the baseline data from the one or more external clinical systems via the communication interface, wherein the baseline data includes electrophysicological signals.

13. The system of claim 11, wherein the processor executes further instructions to receive sensor data from a virtual-reality headset.

14. The system of claim 11, wherein scoring the features includes assigning a higher weight to specific features characteristic of a particular pathological process.

15. The system of claim 11, wherein the processor executes further instructions generate a biomarker profile based on the scored biomarker features.

16. The system of claim 11, wherein the neuromodulation protocol includes one or more virtual-reality environment assets configured to modulate the user.

17. The system of claim 11, wherein the neuromodulation protocol includes one or more neuromodulation parameters based on the determined therapeutic targets.

18. The system of claim 11, wherein the processor executes further instructions to update the neuromodulation protocol based on an analysis of a user response to the provided protocol, wherein one or more neuromodulation parameters are adjusted.

19. The system of claim 11, further comprising tracking the scored features over time to predict a disease progression.

20. A non-transitory, computer-readable storage medium, having embodied thereon a program executable by a processor to perform a method for biomarker-guided personalized neuromodulation therapy delivered within a virtual-reality environment, the method comprising:
storing in memory baseline data of biomarkers associated with one or more neurological conditions;
receiving, via a communication interface, biomarker data associated with a user from one or more external clinical systems;
extracting one or more features from the biomarker data for one or more deviations from the baseline data;
scoring the features based on the deviations, wherein the score is weighted based on indicators of significance;
determining one or more therapeutic targets based on the scored features; and
providing a neuromodulation protocol in the virtual-reality environment based on the determined therapeutic targets.
